(19) United States
(12) Patent Application Publication
(10) Pub. No.: US 2012/0157800 A1
(43) Pub. Date: Jun. 21, 2012

(54) DERMATOLOGY IMAGING DEVICE AND METHOD

(76) Inventor: Jaime A. Tschen, Houston, TX (US)

(21) Appl. No.: 13/246,020

(22) Filed: Sep. 27, 2011

Related U.S. Application Data
(60) Provisional application No. 61/424,336, filed on Dec. 17, 2010.

(51) Int. Cl. A61B 5/00

(52) U.S. Cl. 600/306

(57) ABSTRACT

A medical imaging system that allows collection of current and patient provided historic photographs to compare with current photographs, correct for photographic variables, and provide a directly comparable lesion outline and color map for direct comparison and diagnosis.

### PRIOR RELATED APPLICATIONS

This application claims priority to U.S. Provisional Application 61/424,336, filed Dec. 17, 2010, and incorporated by reference in its entirety.

### FEDERALLY SPONSORED RESEARCH STATEMENT

Not applicable.

### REFERENCE TO MICROFICHE APPENDIX

Not applicable.

### FIELD OF THE INVENTION

The invention relates to medical imaging technology for dermatology, in particular software and systems to allow collection of current and patient provided historic photographs, digital correction for photographic variables, and provide a directly comparable lesion outline and color map for direct comparison and diagnosis.

### BACKGROUND OF THE INVENTION

The dermatoscope or dermoscope is an instrument developed for the evaluation and management of pigmented skin lesions. Non-melanocytic lesions/tumors can also be evaluated with this instrument. A proficient health care professional can minimize false positive diagnoses using such instruments by becoming more accurate in the clinical diagnosis of lesions. Potentially, fewer negative lesions need be biopsied, at the same time more early tumors will undergo a biopsy for early recognition and definitive treatment.

The capability to obtain photographs and store them in a computer as well as in the patients electronic medical records (required by federal law by 2012 for all patients and practices) will make this technique even more appropriate for early recognition of lesions anywhere the patient goes.

Eye scanners have been in use for several years now. These devices identify people by their irises and is considered the 21^{st} century equivalent for fingerprint analysis. Their limitations are mainly with distance and movement. Honeywell has overcome some of these limitations using software that flattens the image and develops a speckle pattern much like a bar code. Similar software is used for documents such as airplane boarding passes. Fingerprints are also analyzed by computers for identification/recognition. Geology surveys also use similar computer analysis for changes in urban and rural areas.

In short, the technology is available to develop a computer software that will recognize a change in a pigmented lesion—and very likely a non-pigmented lesion as well—when an initial image is compared with a subsequent one making the operator know that a particular lesion has changed in color, size, depth, or shape. Change is considered an important, if not the most important, parameter in the diagnosis of pigmented lesions.

Technology has already been developed to address one or more aspects of medical imaging needs. U.S. Pat. No. 7,162,063, for example, discloses a digital skin lesion imaging system that can detect a significant change in skin lesions by providing digital baseline image data of an area of a patient's skin by placing a calibration piece on the area and then positioning a digital camera to frame the area to produce a digital baseline image of the area. The digital baseline image is then processed to provide a partially transparent baseline image, which is then printed on a transparent sheet to produce template. After a time period, the same calibration piece is again place on the same area of the patient's skin, and the template is placed over the viewfinder display of the camera to allow precise framing of the same area. Thereafter, two images can be compared to determine whether the lesion has significantly grown. However, in this patent the step of producing a transparent template and the use of the calibration piece are less desirable because they are less user friendly. Furthermore, the system is only applicable over long term patient treatment, and cannot be used to evaluate historic photographs produced by the patient. Thus, changes that occurred prior to commencing treatment cannot be accurately monitored. In effect, the system is somewhat primitive, amounting to little more than placing a ruler beside a lesion for photographic comparison.

U.S. Pat. No. 7,259,731 discloses a system for overlaying medical images to facilitate detection of lesion changes. Specifically, to enable better alignment of images for comparison, an image registration/comparison engine uses some “relatively stable image feature(s), such as anatomical landmarks” as a basis for aligning multiple images. However, this system is only a mirror based system that allows the physical projection of one image onto another, and thus is quite primitive in concept and implementation, and completely fails to realize the power of digital manipulation of data.

US20090310843, for example, discloses a device for displaying the differences in medical images taken at different times. By employing a position-displacement correcting mechanism to correct the position-displacement of an image, a viewer can more readily compare the difference of the lesion itself instead of position displacement. Specifically, the medical images described in this patent application are tomograms. Tomograms are in black and white with usually two-dimensional reference only, and therefore the correction is much easier than when there is photograph of a patient taken from outside of the body with angles not necessarily perpendicular to the lesion.

US20020150291 provides a method for correcting the color of an image based on known memory color, so as to correct the skin color of a subject in the image due to a defect during recording or the lighting difference. Generally speaking, the method comprises the following steps: at least one pattern area or image pattern is being detected with respect to its presence and its location, and preferably also with respect to its dimensions; an existing color in the at least one detected pattern area or image pattern is being determined; at least one replacement color value (memory color) is being provided, said value being related to the respective at least one pattern area or image pattern and the determined existing color is replaced by said at least one replacement color value, to correct the color in the image pattern or image area. However, this patent addresses only a single aspect of medical imaging needs.

US20070049832 provides a method for medical monitoring and treatment. The method is accomplished by using a scanner to scan the skin of a subject at a close distance to obtain various information, including the reflective properties of skin sections, and morphology of the skin. Through multiple scanning and comparison of information obtained, one can determine whether the skin has a lesion that requires treatment or further medical attention. Specifically, by employing “feature recognition software” the system can define medically relevant attributes from the scanned data, and the features may include cheekbone, nose, ear, etc., that are common to the face recognition software. This patent, like the others, does not allow for use of historic photographs.

U.S. Pat. No. 5,497,430 provides a method for extracting invariable features of a human face despite of difference of images in scale, position or rotation. An in-depth discussion about how that is accomplished is provided therein. This invention is based on a unique combination of a robust face feature extractor and a highly efficient artificial neural network. A real-time video image of a face can serve as the input to a high-speed face feature extractor, which responds by transforming the video image to a mathematical feature vector that is highly invariant under face rotation (or tilt), scale (or distance), and position conditions. This highly invariant mathematical feature representation is believed to be the reason for the extremely robust performance of the invention, and is advantageously capable of the rapid generation of a mathematical feature vector of at least 20 to 50 elements from a face image made up of, for example, 256x256 or 512x512 pixels. This represents a data compression of at least 1000:1. The feature vector is then input into the input neurons of a neural network (NN), which advantageously performs real-time face identification and classification.

U.S. Pat. No. 7,221,809 discloses a method for face recognition by generating a 3-D model of a face from a series of 2-D images. By taking into account of lighting, expression, orientation and other factors to obtain a 3-D face model, face recognition can be accomplished by comparing 2-D images generated from the 3-D model. In this system, the three-dimensional features (such as length of nose, surface profile of chin and forehead, etc.) on a human face can be used, together with its two-dimensional texture information, for a rapid and accurate face identification. The system compares a subject image acquired by surveillance cameras to a database that stores two-dimensional images of faces with multiple possible viewing perspectives, different expressions and different lighting conditions. These two-dimensional face images are produced digitally from a single three-dimensional image of each face via advanced three-dimensional image processing techniques. This method purports to greatly reduce the difficulty for face-matching algorithms to determine the similarity between an input facial image and a facial image stored in the database, thus improving the accuracy of face recognition, and overcoming the orientation, facial expression and lighting vulnerabilities of current two-dimensional face identification algorithms. Additionally, the technology is said to solve the orientation variance and lighting condition variance problems for face identification systems.

However, each of these system address only certain aspects of medical imaging. A truly robust imaging software system would be able to automatically correct for distance and lighting, angle of photograph, age related changes in boney structure and facial expression, as well as the typical changes that are detected in epidermal lesions. The ideal system would be able to collect patient provided photographs, and incorporate these into the patients record, thus allowing accurate comparison of the lesion over a much longer period of time.

### SUMMARY OF THE INVENTION

The invention relates to a truly robust imaging software system, that can automatically correct for age related changes in boney structure and transient facial expressions, distance, angle of photograph, lighting changes, as well as the typical changes that are detected in epidermal lesions. The system allows the collection of patient provided photographs, and their incorporation into the patients record, thus allowing accurate comparison of the lesion over a much longer period of time. The invention also optionally includes the hardware needed to collect the data, manipulate the data as described, and to display and/or store such data, and provides the various user interface modules needed to make the system intuitive, robust and easy to use.

A number of face recognition algorithms have been developed. They are:

Independent Component Analysis (ICA) minimizes both second-order and higher-order dependencies in the input data and attempts to find the basis along which the data (when projected onto them) are—statistically independent. Bartlett et al. provided two architectures of ICA for face recognition task: Architecture I—statistically independent basis images, and Architecture II—factorial code representation.

Evolutionary Pursuit EP. An eigenspace-based adaptive approach that searches for the best set of projection axes in order to maximize a fitness function, measuring at the same time the classification accuracy and generalization ability of the system. Because the dimension of the solution space of this problem is too big, it is solved using a specific kind of genetic algorithm called Evolutionary Pursuit.

Elastic Bunch Graph Matching (EBGM). All human faces share a similar topological structure. Faces are represented as graphs, with nodes positioned at fiducial points. (exes, nose . . . ) and edges labeled with 2-D distance vectors. Each node contains a set of 40 complex Gabor wavelet coefficients at different scales and orientations (phase, amplitude). They are called “jets”. Recognition is based on labeled graphs. A labeled graph is a set of nodes connected by edges, nodes are labeled with jets, edges are labeled with distances. The EGBM is based upon the USC algorithm in the FERET tests.

Kernel methods. The face manifold in subspace need not be linear. Kernel methods are a generalization of linear methods. Direct non-linear manifold schemes are explored to learn this non-linear manifold.

Linear Discriminant Analysis (LDA) finds the vectors in the underlying space that best discriminate among classes. For all samples of all classes the between-class scatter matrix SB and the within-class scatter matrix SW are defined. The goal is to maximize SB while minimizing SW, in other words, maximize the ratio det|SB|/det|SW|.

This ratio is maximized when the column vectors of the projection matrix are the eigenvectors of (SŴ−1×SB).

The Trace transform, a generalization of the Radon transform, is a new tool for image processing which can be used for recognizing objects under transformations, e.g. rotation, translation and scaling. To produce the Trace transform one computes a functional along tracing lines of an image. Different Trace transforms can be produced from an image using different trace functionals.

An Active Appearance Model (AAM) is an integrated statistical model which combines a model of shape variation with a model of the appearance variations in a shape-normalized frame. An AAM contains a statistical model if the shape and gray-level appearance of the object of interest which can generalize to almost any valid example. Matching to an image involves finding model parameters, which minimize the difference between the image and a synthesized model example projected into the image.

3-D Morphable Model. The human face is a surface lying in the 3-D space intrinsically. Therefore the 3-D model should be better for representing faces, especially to handle facial variations, such as pose, illumination etc. Blantz et al. proposed a method based on a 3-D morphable face model that encodes shape and texture in terms of model parameters, and algorithm that recovers these parameters from a single image of a face.

3-D Face Recognition. The main novelty of this approach is the ability to compare surfaces independent of natural deformations resulting from facial expressions. First, the range image and the texture of the face are acquired. Next, the range image is preprocessed by removing certain parts such as hair, which can complicate the recognition process. Finally, a canonical form of the facial surface is computed. Such a representation is insensitive to head orientations and facial expressions, thus significantly simplifying the recognition procedure. The recognition itself is performed on the canonical surfaces.

Bayesian Framework. A probabilistic similarity measure based on Bayesian belief that the image intensity differences are characteristic of typical variations in appearance of an individual. Two classes of facial image variations are defined: intrapersonal variations and extrapersonal variations. Similarity among faces is measures using Bayesian rule.

Given a set of points belonging to two classes, a Support Vector Machine (SVM) finds the hyperplane that separates the largest possible fraction of points of the same class on the same side, while maximizing the distance from either class to the hyperplane. PCA is first used to extract features of face images and then discrimination functions between each pair of images are learned by SVMs.

Hidden Markov Models (HMM) are a set of statistical models used to characterize the statistical properties of a signal. HMM consists of two interrelated processes: (1) an underlying, unobservable Markov chain with a finite number of states, a state transition probability matrix and an initial state probability distribution and (2) a set of probability density functions associated with each state.

Boosting & Ensemble Solutions. The idea behind Boosting is to sequentially employ a weak learner on a weighted version of a given training sample set to generalize a set of classifiers of its kind. Although any individual classifier may perform slightly better than random guessing, the formed ensemble can provide a very accurate (strong) classifier. Viola and Jones build the first real-time face detection system by using AdaBoost, which is considered a dramatic breakthrough in the face detection research. On the other hand, papers by Guo et al. are the first approaches on face recogntion using the AdaBoost methods.

Video-Based Face Recognition Algorithms. During the last couple of years more and more research has been done in the area of face recognition from image sequences. Recognizing humans from real surveillance video is difficult because of the low quality of images and because face images are small. Still, a lot of improvement has been made.

Skin texture analysis. Another emerging trend uses the visual details of the skin, as captured in standard digital or scanned images. This technique, called skin texture analysis, turns the unique lines, patterns, and spots apparent in a person's skin into a mathematical space. Tests have shown that with the addition of skin texture analysis, performance in recognizing faces can increase 20 to 25 percent. Skin texture analysis is expected to be particularly beneficial in correcting for skin distortions that are not facial.

A combination PCA and LDA algorithm based upon the University of Maryland algorithm in the FERET tests.

A Bayesian Intrapersonal/Extrapersonal Image Difference Classifier based upon the MIT algorithm in the FERET tests.

Additional algorithms may be discussed in the patents described above or in the literature, and can also be employed. In particular, free iris recognition software is readily available, see e.g., Iris Recognition System 1.0, which consists of an automatic segmentation system that is based on the Hough transform, and is able to localize the circular iris and pupil region, occluding eyelids and eyelashes, and reflections. The extracted iris region was then normalized into a rectangular block with constant dimensions to account for imaging inconsistencies. Finally, the phase data from 1D Log-Gabor filters was extracted and quantized to four levels to encode the unique pattern of the iris into a bit-wise biometric template.

Likewise, GIRIST (GRUS IRIS TOOL) is a free iris recognition software by GRUSOFT; Iris Recognition Application from The Imperial College of London (projectiris.co.uk/iris); Iris ID; and the like may also prove beneficial, particular in color correction applications, since the iris does not change barring disease or trauma. Further, although being specialized to detect pupils and the unique iris pattern in each individual, such algorithms can be easily adapted to mapping skin lesions instead of eyes.

Each of these algorithms are available, indeed, software downloads are available for many of them. These will be obtained, modified as needed for the indication described and an appropriate user interface for the application designed. The software will then be tested for robustness using existing photographs and the results compared against medical records to ascertain the accuracy of the system.

### BRIEF DESCRIPTION OF THE DRAWINGS

FIG. 1. Schematic showing outline of the system processing steps.

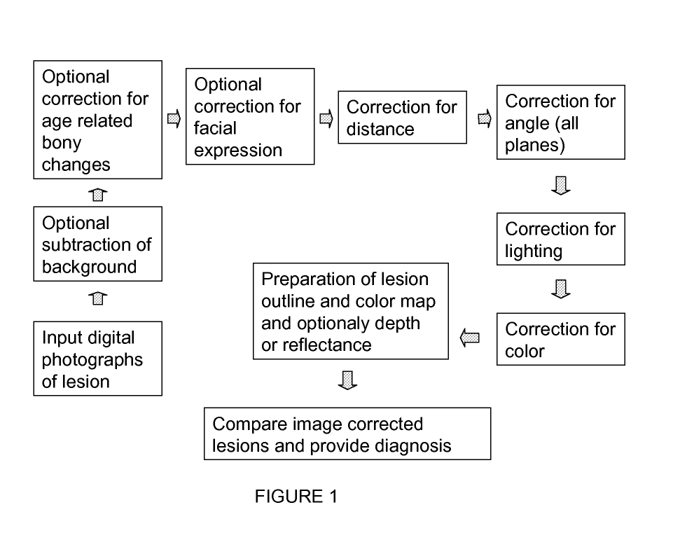

### DESCRIPTION OF EMBODIMENTS OF THE INVENTION

The present invention provides a method for conveniently monitoring a skin lesion by comparing a pre-existing image having at least a portion of the skin lesion with a current image having at least a portion of the skin lesion. Specifically, by employing one or more pattern-recognition algorithm(s) capable of correcting the difference in space, angle or orientation, lighting, as well as differences in age and facial expression, the pre-existing image does not have to be taken by using special equipment under specific conditions. Instead, after processing by the pattern-recognition algorithm(s), the skin-lesion in the pre-existing image is calibrated to the dimension and angles comparable to the current image, thus facilitating the monitoring of the lesion over time.

Alternatively, the present invention allows capturing and identifying certain basic features in the patient-provided images other than the skin lesion of interest, thereafter these basic features are provided as an indicator for capturing and standardizing the current image. Preferably the basic features include those that do not or only slightly change with age. For example, in a patient-provided image showing the skin lesion that also shows both eyes, a current image can be taken to include those eyes, so that the distance between the eyes, which does not change with age (except in the young), can be an indicator to standardize and/or calibrate images in order to perform lesion-comparison.

In more detail, the system first captures and if necessary digitizes old and current patient photographs. The camera and lighting can be any kind of camera and lighting system, but is preferably a digital camera, and well lit environment, that seeks to minimize shadows.

The system can easily be adapted to whole body imaging, and camera arrays can be used instead of single camera photography. Simple, yet powerful camera systems can be used, e.g., the now ubiquitous phone cameras, and can also be combined with magnifiers. Indeed, an i-phone app already exists for such a use.

Where necessary, corrections are made to compensate for age related differences using existing algorithms to project (or subtract) age related boney changes to the skeletal structure. However, this is only required where the photographs span the early growth periods (e.g., puberty) and thus will only rarely be required.

Also, corrections may be needed to accommodate e.g., facial expressions, e.g., for lesions near the mouth that can be stretched when a patient smiles. The 2D image of the patient face can be mapped onto a 3D structure, and such changes adjusted for in the 3D model. By correcting for “facial expressions” herein we imply that any distortion caused by the underlying muscular or boney structure can be corrected for. Thus, the skin over the biceps may be distorted when the biceps are tightened, but these superficial changes can be corrected for using the same software that corrects facial expressions.

The photographs are also adjusted to correct for angle, distance, and facial expressions based, for example on existing 3D facial recognition software. Several systems are available to this sort of complicated mapping, and any of the existing systems may be suitable, particularly where speed is not as essential in a medical as opposed to security environment. Generally speaking, however, the systems measure common parameters; such as distances and angle between fixed features and then extrapolate that data from a 2D photograph to a 3D model.

Next, lighting, color and shadows can be corrected, for example, based on parameters that do not vary significantly over time such as eye color (assuming no loss of sight or cataracts) or hair or teeth color, or combinations thereof.

Image recognition software can then produce an outline of the lesion of interest and/or a color map of the lesion, and the two outlines, color map (and in some instances depth map) can be compared for purposes of detecting change to the outline, color or depth of the lesion for diagnostic purposes. The two maps can be overlaid and visually compared, but the software can also prepare a difference map, whereby only differences are shown, or the differences are highlighted for example in a contrasting color. If desired, the map can be mathematically flattened for visualization purposes, or if preferred the lesion map can be visualized with the existing 3D architecture.

Additionally, the present invention can include the feature of database searching and preliminary diagnosis. More specifically, by connecting to existing dermatology databases and providing characteristics of the skin lesion (such as the location, growth rate, shape, color, etc.) and/or the images, database searching can be performed, and if possible matches are found, a preliminary diagnosis can be provided for the dermatologist's review.

The body part to which the present invention is applicable is not limited, as long as calibration/standardization/comparison between the patient-provided image and a current image is viable. Theoretically human faces provide the most features to be readily recognizable, but other body parts can also be the subject of comparison.

In one embodiment of the present invention, the method also comprises the step of performing a side-by-side or overlapping image comparison between the adjusted pre-existing image and the adjusted current image so as to facilitate the determination of any change of the skin lesion. Preferably the side-by-side or overlapping image comparison is displayed on a screen or can be printed out for later storage of the comparison. In one embodiment, the image comparison can be saved for follow-up purposes in the future.

The invention thus provides the software needed to effect the various calibrations and adjustments, together with a user friendly interface. In some embodiments, the system also includes the camera and lighting needed to take current photographs, but this is not essential and it is specifically an intent of this invention to allow the practitioner to collect a range of patient produced photographs so that the doctor can follow a lesions over time, even before the patient sought medical assistance.

Another aspect of the invention is the database for storing adjusted original and figures, and optionally an interface needed to allow convenient access to same.

### Example 1

We will design and test each component module of the software system independently, as well as their functionality as a whole, and at the same time design and implement a user friendly interface.

### Example 2

We will test the system on a collection of photographs taken over time by medical practitioners, as well as including patient provided photographs and comparing the generated results with the software designed in Example 1 with patient records to see which lesions were in fact biopsied and determined to be problematic.

Although exemplified herein from still photographs, the algorithms can easily be applied to video footage as well, which can be considered a very large collection of stills. However, traditional stills are currently preferred because video images have historically been of lower quality.

The following articles are incorporated by reference herein in their entirety.
M. Turk, A. Pentland, Eigenfaces for Recognition, Journal of Cognitive Neurosicence, Vol. 3, No. 1, Win. 1991, pp. 71-86
P. N. Belhumeur, J. P. Hespanha, D. J. Kriegman, Eigenfaces vs. Fisherfaces: Recognition Using Class Specific Linear Projection, IEEE Transactions on Pattern Analysis and Machine Intelligence, Vol. 19, No. 7, July 1997, pp. 711-720
A. K. Jain, R. P. W. Duin, J. Mao, Statistical Pattern Recognition: A Review, IEEE Transactions on Pattern Analysis and Machine Intelligence, Vol. 22, No. 1, January 2000, pp. 4-37
M.-H. Yang, D. J. Kriegman, N. Ahuja, Detecting Faces in Images: A Survey, IEEE Transactions on Pattern Analysis and Machine Intelligence, Vol. 24, No. 1, January 2002, pp. 34-58
R. Chellappa, C. L. Wilson, S. Sirohey, Human and Machine Recognition of Faces: A Survey, Proceedings of the IEEE, Vol. 83, Issue 5, May 1995, pp. 705-740
P. J. Phillips, H. Moon, S. A. Rizvi, P. J. Rauss, The FERET Evaluation Methodology for Face-Recognition Algorithms, IEEE Transactions on Pattern Analysis and Machine Intelligence, Vol. 22, No. 10, October 2000, pp. 1090-1104
W. Zhao, R. Chellappa, P. J. Phillips, A. Rosenfeld, Face Recognition: A Literature Survey, ACM Computing Surveys, Vol. 35, No. 4, 2003, pp. 399-458
L. Wiskott, J.-M., Fellous, N. Kruger, C. D. Von Malsburg, Face Recognition by Elastic Bunch Graph Matching, IEEE Transactions on Pattern Analysis and Machine Intelligence, Vol. 19, No. 7, July 1997, pp. 775-779
V. Bruce, A. Young, Understanding Face Recognition, The British Journal of Psychology, Vol. 77, No. 3, August 1986, pp. 305-327
P. Viola, M. J. Jones, Robust Real-Time Face Detection, International Journal of Computer Vision, Vol. 57, No. 2, 2004, pp. 137-154
R. Brunelli, T. Poggio, Face Recognition: Features versus Templates, IEEE Transactions on Pattern Analysis and Machine Intelligence, Vol. 15, No. 10, October 1993, pp. 1042-1052
M. Kirby, L. Sirovich, Application of the Karhunen-Loeve Procedure for the Characterization of Human Faces, IEEE Transactions on Pattern Analysis and Machine Intelligence, Vol. 12, No. 1, 1990, pp. 103-108
J. Sergent, S. Ohta, B. MacDonald, Functional Neuroanatomy of Face and Object Processing, A Positron Emission Tomography Study, Brain, Vol. 115, No. 1, February 1992, pp. 15-36
S. Bentin, T. Allison, A. Puce, E. Perez, G. McCarthy, Electrophysiological Studies of Face Perception in Humans, Journal of Cognitive Neuroscience, Vol. 8, No. 6, 1996, pp. 551-565
B. Moghaddam, A. Pentland, Probabilistic Visual Learning for Object Representation, IEEE Transactions on Pattern Analysis and Machine Intelligence, Vol. 19, No. 7, July 1997, pp. 696-710
R. Diamond, S. Carey, Why Faces Are and Are Not Special. An Effect of Expertise, Journal of Experimental Psychology: General, Vol. 115, No. 2, 1986, pp. 107-117
J. W. Tanaka, M. J. Farah, Parts and Wholes in Face Recognition, Quarterly Journal of Experimental Psychology Section A: Human Experimental Psychology, Vol. 46, No. 2, 1993, pp. 225-245
D. L. Swets, J. J. Weng, Using Discriminant Eigenfeatures for Image Retrieval, IEEE Transactions on Pattern Analysis and Machine Intelligence, Vol. 18, No. 8, 1996, pp. 831-836

What is claimed is:

1. A method for detecting skin lesion changes comprising:
obtaining a pre-existing image of a patient showing at least a portion of a skin lesion;
obtaining a current image of the patient showing at least a portion of said skin lesion;
correcting the pre-existing image and the current image by using an image-correction module that: i) optionally corrects for age related bony growth changes, ii) optionally corrects for facial expression or other skin distortions, and corrects for iii) distance, iv) lighting, v) color, and vi) angle of photograph, thus preparing an adjusted pre-existing image and an adjusted current image, and
determining the difference in the skin lesion between the adjusted pre-existing and adjusted current images.

2. The method of claim 1, wherein determining the difference between in the skin lesion between the adjusted pre-existing and adjusted current images requires preparing and comparing an outline and color map of the lesion and detecting differences therein.

3. The method of claim 1, wherein the differences are identified in contrasting color.

4. The method of claim 1, wherein the backgrounds are first subtracted from the preexisting image and the current image.

5. The method of claim 1, wherein the image correction module uses an algorithm selected from Independent Component Analysis (ICA); Eigenspace-based approach; Evolutionary Pursuit (EP); Elastic Bunch Graph Matching (EBGM); Kernel methods; Linear Discriminant Analysis (LDA); Trace Transform; Active Appearance Model (AAM); 3-D Morphable Model; 3-D Face Recognition; Bayesian Framework; Support Vector Machine (SVM); Hidden Markov Models (HMM); Boosting & Ensemble Solutions; Video-Based Face Recognition Algorithms; Skin texture analysis; combination PCA and LDA algorithm; Bayesian Intrapersonal/Extrapersonal Image Difference Classifier, or combinations thereof.

6. The method of claim 1, further comprising displaying i) the adjusted pre-existing image and ii) the adjusted current image and a third image highlighting the differences between i) and ii) in a contrasting color.

7. The method of claim 1, where said differences include differences in color, size, shape, depth, and refractivity.

8. A method for detecting skin lesion changes comprising:
obtaining a pre-existing image of a patient showing at least a portion of a skin lesion;
obtaining a current image of the patient showing at least a portion of said skin lesion;
correcting the pre-existing image and the current image by using an image-correction module that: i) optionally corrects for age related bony growth changes, ii) optionally corrects for facial expression or other skin distortions, and corrects for iii) distance, iv) lighting, v) color, and vi) angle of photograph, thus preparing an adjusted pre-existing image and an adjusted current image,
determining the difference in the skin lesion between the adjusted pre-existing and adjusted current images, and
displaying said differences,
wherein the image correction module uses one or more algorithm(s) selected from Independent Component Analysis (ICA); Eigenspace-based approach; Evolutionary Pursuit (EP); Elastic Bunch Graph Matching (EBGM); Kernel methods; Linear Discriminant Analysis (LDA); Trace Transform; Active Appearance Model (AAM); 3-D Morphable Model; 3-D Face Recognition; Bayesian Framework; Support Vector Machine (SVM); Hidden Markov Models (HMM); Boosting & Ensemble Solutions; Video-Based Face Recognition Algorithms; Skin texture analysis; combination PCA and LDA algorithms; Bayesian Intrapersonal/Extrapersonal Image Difference Classifier, or combinations thereof, and
wherein said differences include at least three differences selected from differences in color, size, shape, depth, and refractivity.
